(19) United States
(12) Patent Application Publication
(10) Pub. No.: US 2023/0218626 A1
(43) Pub. Date: Jul. 13, 2023

(54) METHODS AND COMPOSITIONS TO SUPPORT THE GROWTH OR MAINTENANCE OF OXYGEN-SENSITIVE BACTERIA IN THE GASTROINTESTINAL TRACT

(71) Applicant: DSM IP Assets B.V., Heerlen (NL)

(72) Inventors: Giorgio GABARRINI, Groningen (NL); Hermanus Jozef Martinus HARMSEN, Groningen (NL); Mehdi SADAGHIAN SADABAD, Groningen (NL); Jan Maarten VAN DIJL, Groningen (NL)

(21) Appl. No.: 18/125,002

(22) Filed: Mar. 22, 2023

Related U.S. Application Data
(62) Division of application No. 16/972,470, filed on Dec. 4, 2020.

(30) Foreign Application Priority Data
Jun. 8, 2018 (EP) 18176697.3

(51) Int. Cl. A61K 31/375; A61K 31/525; A61K 9/20; A61P 1/14

(52) U.S. Cl. CPC A61K 31/375; A61K 31/525; A61K 9/20; A61P 1/14

(57) ABSTRACT

Methods and compositions to support the growth or maintenance of oxygen-sensitive bacteria in the gastrointestinal tract of an animal, preferably a mammal, are provided. Particularly, the methods for selectively enhancing the growth of beneficial anaerobic butyrate-producing bacteria, such as Faecaliumbacterium prausnitzii (F. prausnitzii).

### CROSS-REFERENCE TO RELATED APPLICATIONS

This application is a divisional of U.S. application Ser. No. 16/972,470 filed on Dec. 4, 2020 (now abandoned), which in turn is the U.S. national phase of International Application No. PCT/EP2019/064781 filed Jun. 6, 2019 which designated the U.S. and claims priority to EP 18176697.3 filed Jun. 8, 2018, the entire contents of each of which are hereby incorporated by reference.

### FIELD

The present invention relates to methods and compositions to support the growth or maintenance of oxygen-sensitive bacteria in the gastrointestinal tract of an animal, preferably a mammal. Particularly, the invention relates to means and methods for selectively enhancing the growth of beneficial anaerobic butyrate-producing bacteria, such as Faecaliumbacterium prausnitzii (F. prausnitzii).

### BACKGROUND AND SUMMARY

A normal gastrointestinal microbiota is important to prevent inflammatory disorders such as inflammatory bowel disease and other diseases related to microbiota dis-balance.

Probiotics are microorganisms that are claimed to provide health benefits when consumed by maintaining a normal host gastrointestinal microbiota. Probiotic formulations have been used as a dietary supplement for many years. So far, many different probiotic strains and combinations thereof exist, but nearly all of them employ relatively-oxygen tolerant strains for instance Bifidobacterium sp., Lactobacillus sp. and Saccharomyces sp.

Prebiotics are typically compounds that pass undigested through the upper part of the gastrointestinal tract and stimulate the growth or activity of advantageous bacteria that colonize the large bowel by acting as substrate for them. Typical examples of known prebiotics are oligosaccharides, such as fructooligosaccharides, galactooligosaccharides and inulin. Synbiotics refer to nutritional supplements combining probiotics and prebiotics in a form of synergism.

Numerous probiotics, prebiotics and symbiotic formulations are known in the art to maintain or stimulate the level of beneficial oxygen-tolerant bacteria in the gut. In contrast, very few studies have dealt with stimulating oxygen-sensitive bacteria.

Recent research in gut microbiology explores new horizons for pre- and probiotic applications, such as anti-inflammatory treatments including treatment of Crohn's disease. Beneficial bacteria including F. prausnitzii typically utilize a variety of carbohydrates and produce butyrate as a major fermentative end product. Butyrate is well known for its role in promoting and maintaining gut health.

In fact, F. prausnitzii was found to exhibit anti-inflammatory effects on cellular and colitis models, partly due to secreted metabolites able to block NF-κB activation and IL-8 production. F. prausnitzii is one of the most abundant human colon bacteria with numbers ranging from 5-20% of the total microbiota in stools of healthy individuals. It was found that a reduction of F. prausnitzii is associated with a higher risk of postoperative recurrence of Crohn's Disease. The current idea is that counterbalancing dysbiosis including low levels of F. prausnitzii is a promising strategy for treatment of inflammatory bowel diseases such as Crohn's Disease or any other diseases related to microbiota dysbiosis by preventing reoccurrence of exacerbations.

Inflammatory Bowel Disease (IBD) and Irritable Bowel Syndrome (IBS) are a chronic and debilitating illnesses. It is characterized by chronic intestinal inflammation that often shows an intermittent course with acute attacks followed by periods of remission. Clinical symptoms during acute attacks include diarrhea, bleeding, abdominal pain, fever, joint pain, and weight loss. These symptoms can range from mild to severe, and may gradually and subtly develop from an initial minor discomfort, or may present themselves suddenly in full-blown form. IBD can manifest itself in a variety of forms, the most common of which are Crohn's disease and ulcerative colitis. Both of these diseases are similar in terms of clinical symptoms, even though their inflammation patterns are distributed differently in the GI tract. Crohn's disease is a chronic transmural inflammation of the bowel, which can affect the whole gastrointestinal tract, usually in a discontinuous pattern. The initial location of CD is most commonly in the lower ileum. From here the inflammation typically spreads towards proximal parts of the small intestine. However, the colon is also of-ten involved.

Ulcerative colitis is a chronic inflammatory bowel disease affecting only the colon and shows a continuous distribution in the gastrointestinal mucosa. In most patients the focal point of the inflammation is in the distal part of the colon and the rectum. From this origin, the inflammation often spreads proximally. In the most severe cases, the whole colon is affected which is called as “pancolitis”. About 30% of patients suffer from this severe form of UC.

F. prausnitzii and some other strict anaerobe bacteria needed for the afore-mentioned anti-inflammatory and other beneficial effects are extremely sensitive to oxygen and cannot survive an exposure to ambient air for more than a few minutes. As a consequence, probiotic compositions containing F. prausnitzii have not been described thus far despite their promising therapeutic application.

Recognizing the therapeutic potential of beneficial anaerobic bacteria, the present inventors set out to identify new means and methods to enhance the population of butyrate-producing anaerobic bacteria including F. prausnitzii in the gastrointestinal tract. In particular, they aimed at providing novel nutritional ingredient formulations for selectively stimulating butyrate-producing anaerobic bacteria, preferably F. prausnitzii.

It is known i.e. that the intake of riboflavin (also known as vitamin B2) has a positive effect on the gut microbiota (maintaining a “normal” gut microbiota or treating gut microbiota, which is “in bad shape”).

Now, it was surprisingly found that a combination of riboflavin (also known as vitamin B2) and vitamin C (also known as ascorbic acid) in a very specific ratio, is capable of increasing the concentration of F. prausnitzii.

This results in a surprisingly positive effect on the gut microbiota. This is very helpful in treating and/or preventing IBD (such as Crohn's disease and ulcerative colitis)

### BRIEF DESCRIPTION OF THE DRAWINGS

FIG. 1 is a graph showing the effect of both vitamin C and vitamin B2 on Faecalibacterium prausnitzii separately in in vitro experiments, and

FIG. 2 is a graph showing the effect on a co-culture of E. coli and F. prausnitzii in in vitro experiments.

### DETAILED DESCRIPTION

Therefore the present invention relates to a method for the selective growth of F. prausnitzii in the gastrointestinal tract in an animal, comprising administering to the animal a combination of
(i) vitamin C,
(ii) riboflavin, riboflavin phosphate and/or a physiologically acceptable salt thereof, and
   wherein the ratio of (i):(ii) is between 1:1 to 50:1

Furthermore, the present invention relates to a composition comprising a combination of
(i) vitamin C,
(ii) riboflavin, riboflavin phosphate and/or a physiologically acceptable salt thereof, and
   wherein the ratio of (i):(ii) is between 1:1 to 50:1.

Furthermore, the present invention relates to a pharmaceutical composition comprising a combination of
(i) vitamin C,
(ii) riboflavin, riboflavin phosphate and/or a physiologically acceptable salt thereof, and
   wherein the ratio of (i):(ii) is between 1:1 to 50:1 and at least one therapeutically inert carrier.

Furthermore, the present invention relates to a pharmaceutical composition comprising a combination of
(i) vitamin C,
(ii) riboflavin, riboflavin phosphate and/or a physiologically acceptable salt thereof, and
   wherein the ratio of (i):(ii) is between 1:1 to 50:1 for the use as therapeutically active substance for the treatment or prophylaxis of IBD and/or IBS or any other diseases related to microbiota dysbiosis.

Furthermore, the present invention relates to the use of a pharmaceutical composition comprising a combination of
(i) vitamin C,
(ii) riboflavin, riboflavin phosphate and/or a physiologically acceptable salt thereof, and
   wherein the ratio of (i):(ii) is between 1:1 to 50:1 for the use as therapeutically active substance for the treatment or prophylaxis of IBD and/or IBS or any other diseases related to microbiota dysbiosis.

Furthermore, the present invention relates to the use of a pharmaceutical composition comprising a combination of
(i) vitamin C,
(ii) riboflavin, riboflavin phosphate and/or a physiologically acceptable salt thereof, and
   wherein the ratio of (i):(ii) is between 1:1 to 50:1 and at least one and at least one therapeutically inert carrier for the preparation of a medicament for the treatment or prophylaxis of IBD and/or IBS or any other diseases related to microbiota dysbiosis.

Furthermore the present invention relates to a method for the treatment or prophylaxis of IBD and/or IBS or any other diseases related to microbiota dysbiosis, which method comprises administering a therapeutically effective amount of a pharmaceutical combination a combination of
(i) vitamin C,
(ii) riboflavin, riboflavin phosphate and/or a physiologically acceptable salt thereof, and
   wherein the ratio of (i):(ii) is between 1:1 to 50:1.

All the ratios are related to the amount (weight).

The overall amount consumption of vitamin C is around 1 g vitamin C/day. Due to the fact, that there is no recommended daily intake for Vitamin C and Vitamin B2, the amount could also be higher.

It is obvious that the amount could also be lower.

The amount in a formulation, which is consumed by a patient, depends on the galenical formulation (it can be any formulation, preferred are solid formulation, like a tablet, a granule, powder, etc). This means a tablet should not be too big to be swallowed and it should also be possible to formulate the amount in a stable application form.

Riboflavin, also known as vitamin B2, is a micronutrient with a key role in maintaining health in humans and other mammals. It is the central component of the cofactors FAD and FMN, and is therefore required by all flavoproteins. As such, riboflavin is required for a wide variety of cellular processes. It plays a key role in energy metabolism, and for the metabolism of fats, ketone bodies, carbohydrates, and proteins. Riboflavin is found naturally in asparagus, popcorn, bananas, persimmons, okra, chard, cottage cheese, milk, yogurt, meat, eggs, fish, and green beans. Other sources specify cheese, leafy green vegetables, liver, kidneys, legumes, tomatoes, yeast, mushrooms, and almonds.

Vitamin C (also known as ascorbic acid or salts thereof) is commonly used as antioxidant food additives.

The vitamin C can be natural (extracted from a plant/fruit) or it can be synthetic one (chemically or biochemically produced). It is clear also mixture of both can be used.

In the context of the present invention, the intake of riboflavin and the vitamin C can take place in such ways that
(a) a mixture of both compounds can be consumed or
(b) both compounds can be consumed individually (the sequence is not crucial)

As said before component (i) and component (ii) can also be a mixture of different forms (i.e. different salts of the components).

In case the riboflavin and the vitamin C are eaten individually, this should take place in a short period of time.

The easiest (and most convenient) way is that a mixture of the (i) and (ii) are consumed.

This can happen any form. This could be a powder of both or the two ingredients (or mixture of the ingredients) can be formulated. They can be incorporated in any desirable formulation suitable for oral intake. The formulation can be liquid or solid. Preferably, it is a food product, pharmaceutical composition, food or dietary supplement.

The invention provides a method for the selective growth of F. prausnitzii in the gastrointestinal tract in an animal, e.g. a mammal, in need thereof, comprising administering to the animal a mixture of vitamin C and riboflavin, riboflavin phosphate or a physiologically acceptable salt thereof, in an amount effective to selectively stimulate the growth of F. prausnitzii in the gastrointestinal tract.

The animal can be a human, pet or livestock. Thus, veterinary use of the present invention is also encompassed.

Preferably, the animal is a human subject. In one embodiment, the human subject is suffering from an inflammatory gastrointestinal disease, in particular Crohn's disease or a related colitis, or any other diseases related to microbiota dysbiosis. Accordingly, also provided is a method for preventing, treating or reducing the symptoms associated with an inflammatory gastrointestinal disorder, comprising administering to a subject in need thereof an amount of riboflavin effective to maintain, support or stimulate the growth of F. prausnitzii in the gastrointestinal tract. Individuals with exemplary inflammatory gastrointestinal disorders, who may benefit from increasing F. prausnitzii numbers in the GI tract by riboflavin, include patients with Crohn's disease, inflammatory bowel disease and ulcerative colitis. Also encompassed is the treatment of other diseases, conditions or disorders where patients benefit from restoring or increasing F. prausnitzii numbers in the GI tract.

As stated above the gastrointestinal disorders are Crohn's disease, inflammatory bowel disease and ulcerative colitis or any other diseases related to microbiota dysbiosis.

As stated above the formulations, which are described in this patent application can also be in a galenical form.

The galenical formulation can comprise any pharmaceutically acceptable auxiliary agents, which are necessary, needed or desired to form such a galencial formulation.

The galencial formulation can be in any form, which is suitable for patients. Most commonly it is in a solid form (such as a tablet, powder or similar).

By “pharmaceutically acceptable” is meant a material that is not biologically or otherwise undesirable.

Pharmaceutically acceptable excipients include but are not limited to binders, diluents, lubricants, glidants and surface-active agents.

Such pharmaceutically acceptable excipients are used when Mesalamine and riboflavin are integrated into a suitable form for administration.

The amount of additive employed will depend upon how much active agent is to be used. One excipient can perform more than one function.

Binders include, but are not limited to, starches such as potato starch, wheat starch, corn starch; microcrystalline cellulose; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone and the like and mixtures thereof.

Fillers or diluents, which include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as Mg, Al or Ca or Zn stearate, polyethylene glycol, glyceryl behenate, mineral oil, sodium stearyl fumarate, stearic acid, hydrogenated vegetable oil and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

The pharmaceutical formulation according to the present invention include but is not limited to tablets (single layered tablets, multilayered tablets, MUPS, mini tablets, bioadhesive tablets, caplets, matrix tablets, tablet within a tablet, mucoadhesive tablets, modified release tablets, pulsatile release tablets, timed release tablets), pellets, beads, granules, sustained release formulations, capsules, microcapsules, tablets in capsules and microspheres, matrix formulations, microencapsulation and powder/pellets/granules for suspension.

The galenical formulation of the invention can optionally have one or more coatings such as film coating, sugar coating, enteric coating, bioadhesive coating and other coatings known in the art. These coatings help pharmaceutical formulations to release the drug at the required site of action. In one example, the additional coating prevents the dosage from contacting the mouth or esophagus. In another example, the additional coating remains intact until reaching the small intestine (e.g., an enteric coating). Premature exposure of a bioadhesive layer or dissolution of a pharmaceutical dosage form in the mouth can be prevented with a layer or coating of hydrophilic polymers such as HPMC or gelatin. Optionally, Eudragit FS 3OD or other suitable polymer may be incorporated in coating composition to retard the release of the drug to ensure drug release in the colon.

These coating layers comprises one or more excipients selected from the group comprising coating agents, opacifiers, taste-masking agents, fillers, polishing agents, coloring agents, antitacking agents and the like.

The galenical formulations of the invention can be coated by a wide variety of methods. Suitable methods include compression coating, coating in a fluidized bed or a pan and hot melt (extrusion) coating. Such methods are well known to those skilled in the art.

Non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, triethyl citrate, triacetin, etc.

Also, coatings of polymers that are susceptible to enzymatic cleavage by colonic bacteria are another means of ensuring release to distal ileum and ascending colon. Materials such as calcium pectinate can be applied as coatings to dosage form and multiparticulates and disintegrate in the lower gastrointestinal tract, due to bacterial action. Calcium pectinate capsules for encapsulation of bioadhesive multiparticulates are also available.

In embodiments of the present invention, a pharmaceutical combinations of Mesalamine or a pharmaceutically acceptable salt or prodrugs thereof and riboflavin and at least one swellable polymer. Swellable polymers include, but are not limited to, a crosslinked poly(acrylic acid), a poly(alkylene oxide), a polyvinyl alcohol), a polyvinyl pyrrolidone); a polyurethane hydrogel, a maleic anhydride polymer, such as a maleic anhydride copolymer, a cellulose polymer, a polysaccharide, starch, and starch based polymers.

The pharmaceutical compositions of the present invention can optionally include one or more solubilizers, i.e., additives to increase the solubility of the pharmaceutical active ingredient or other composition components in the solid carrier. Suitable solubilizers for, use in the compositions of the present invention include: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide, and polyvinylpyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, .delta.-valerolactone and isomers thereof, .beta.-butyrolactone and isomers thereof; and other solubilizers known in the art, such as dimethyl acetamide, dimethyl isosorbide (Arlasolve DMI (ICI)), N-methyl pyrrolidones (Pharmasolve (ISP)), monooctanoin, diethylene glycol monoethyl ether (available from Gattefosse under the trade name Transcutol), and water.

Preferred solubilizers include triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-600, glycofurol, transcutol, propylene glycol, and dimethyl isosorbide. Particularly preferred solubilizers include sorbitol, glycerol, triacetin, ethyl alcohol, SLS, polyethylene glycols glycofurol and propylene glycol. Cyclodextrins polyoxomers, surfactants and like

All formulations as well as the Galenical formulation described and disclosed above can be produced by using well-known methods and processes.

### Example

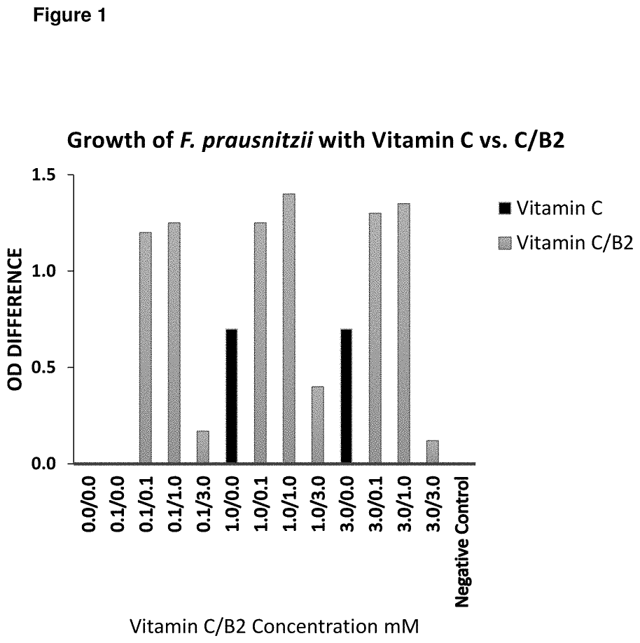

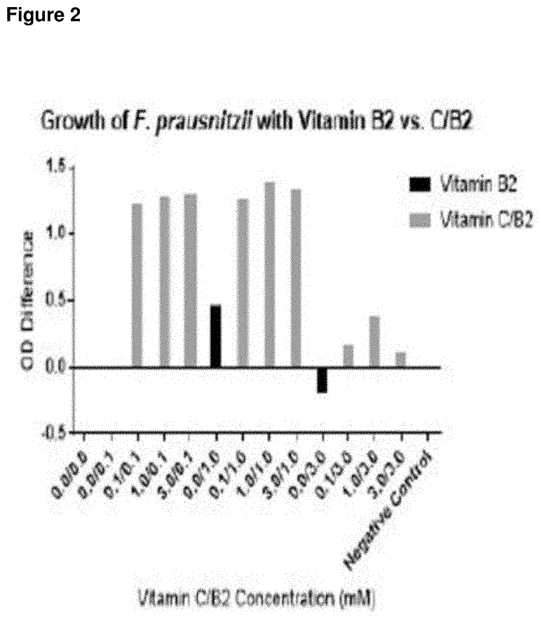

In in vitro experiments, the effect of both vitamin C and vitamin B2 on Faecalibacterium prausnitzii separately (FIG. 1) as well as on a co-culture of E. coli and F. prausnitzii (FIG. 2); was studied. Inoculation of F. prausnitzii was in the anaerobic chamber and E. coli was aerobically inoculated. All the growth were aerobic (outside of anaerobic chamber). Optical densities (ODs) were measured before (t=0 h) and after (t=24 h) incubation. Medium broths with a maximum OD of 2 at t=0 h were excluded from analysis. Differences in OD at t=0 h and t=24 h were graphed to see changes in growth after vitamin intervention. To quantify the growth of each strain in the aerobic co-cultures, a FISH analysis was performed.

For F. prausnitzii, the increase in aerobic growth was larger after addition of a combination of vitamin C with low vitamin B2 (0.1 and 1.0 mM) concentrations compared to only vitamin C or vitamin B2, or combination of vitamin C and higher vitamin B2 concentrations (3.0 mM) (FIG. 1).

All F. prausnitzii and E. coli co-culture samples showed a significantly increased percentage of F. prausnitzii compared to controls after addition of different vitamin concentration. The highest ratio was with 3.0/0.1 mM (30:1) of vitamin C/Vitamin B2 (FIG. 2).

1. A method for the treatment or prophylaxis of Inflammatory Bowel Disease (IBD) and/or Irritable Bowel Syndrome (IBS) or any other diseases related to microbiota dysbiosis, the method comprising administering to an animal subject in need of treatment or prophylaxis of IBD and/or IBS a pharmaceutical composition consisting of a combination of the following components:
(i) vitamin C,
(ii) riboflavin, riboflavin phosphate and/or a physiologically acceptable salt thereof, and
(iii) at least one therapeutically inert carrier, wherein
the components (i) and (ii) are present in amounts to obtain a weight ratio of (i):(ii) of between 1:1 to 50:1 sufficient to selectively enhance growth of Faeca-liumbacterium prausnitzii in a gastrointestinal tract of the animal subject.

2. The method according to claim 1, wherein the method comprises administering the pharmaceutical composition in a solid form.

3. The pharmaceutical composition according to claim 2, wherein the solid form of the pharmaceutical composition is a tablet.
